(19) United States
(12) Patent Application Publication
(10) Pub. No.: US 2023/0270939 A1
(43) Pub. Date: Aug. 31, 2023

(54) Improvements of the Usability of a Drug Delivery Device

(71) Applicant: Sanofi, Paris (FR)

(72) Inventor: Stephan Riedel, Frankfurt am Main (DE)

(21) Appl. No.: 18/015,407

(22) PCT Filed: Jul. 14, 2021
(86) PCT No.: PCT/EP2021/069547

§ 371 Date: Jan. 10, 2023

(30) Foreign Application Priority Data
Jul. 15, 2020 (EP) 20315349.9

(51) Int. Cl. A61M 5/172; A61M 5/32; G16H 40/63; G16H 20/17

(52) U.S. Cl. CPC A61M 5/172; A61M 5/3202; G16H 40/63; G16H 20/17; A61M 2205/583; A61M 2205/502

(57) ABSTRACT

A system includes a drug injection device including a distal end and a proximal end that includes a syringe for injecting a drug into a patient's body. The system further includes an electronic module for wirelessly exchanging data related to a usage of the drug injection device with a device paired with the electronic module, the electronic module being attached to or integrated in the distal end of the drug injection device; a computing device configurable for a pairing with the electronic module for wireless exchange of the data and including a touch interface; a touch input means for providing inputs on the touch interface, the touch input means attached to the drug injection device; and a computer program including instructions for execution by a processor of the computing device to configure the computing device for the pairing with the electronic module based on the inputs received via the touch interface.

### CROSS REFERENCE TO RELATED APPLICATIONS

The present application is the national stage entry of International Patent Application No. PCT/EP2021/069547, filed on Jul. 14, 2021, and claims priority to Application No. EP 20315349.9, filed on Jul. 15, 2020, the disclosures of which are incorporated herein by reference.

### TECHNICAL FIELD

The present disclosure relates to improvements of the usability of a drug delivery device.

### BACKGROUND

A variety of diseases exists that require regular treatment by delivery, particularly injection of a drug or medicament. Such injection can be performed by using injection devices, which are applied either by medical personnel or by patients themselves.

Injection devices for use by patients themselves are for example the disposable Solostar™ injection pen and the reusable Allstar™ injection pen, both from Sanofi. The Allstar™ injection pen is described in detail in the international patent application WO2014/033195A1. An injection pen such as the Solostar™ and Allstar™ injection pens comprises a distal and a proximal end, wherein in the context of this disclosure the term proximal refers to the direction pointing towards the patient during an injection while the term distal refers to the opposite direction pointing away from the patient.

Drug injection devices for usage by patients themselves may be equipped with electronics for measuring and storing data related to the usage. The usage related data may also be transmitted via a wireless link or a wireline connection to an external device such as a smartphone, a tablet or laptop computer, or in the cloud. For example, US 2019/0134305 A1 discloses a medication delivery device, for example an injection pen or a wearable pump, which can be paired with an external device for providing data captured from a flow sensor relating to medicine delivery to a patient to a paired external device. The device can have Bluetooth® communication and/or near field communication (NFC) circuits for proximity-based pairing and connectivity with the external device for real-time or deferred transfer of captured data to the external device.

### SUMMARY

This disclosure describes improvements of the usability of a drug delivery device, particularly of an injection pen equipped with electronics for transmitting data related to the usage of the pen to an external device via a wireless communication connection requiring a pairing of the external device with the electronics.

In one aspect the present disclosure provides a system comprising a drug injection device having a pen-like shape with a distal and a proximal end, the proximal end comprising a syringe for injecting a drug into a patient's body, an electronic module for wireless exchange of data related to a usage of the drug injection device with a device paired with the electronic module, the electronic module being attached to or integrated in the distal end of the drug injection device, a computing device being configurable for pairing with the electronic module for wireless exchange of the data related to the usage of the drug injection device and comprising a touch interface, touch input means attached to the drug injection device and provided for making inputs on the touch interface of the computing device, and a computer program comprising instructions for execution by a processor of the computing device to configure the computing device for the pairing with the electronic module based on the inputs received via the touch interface. The touch input means can e.g. be attached to the proximal end of the drug injection device. This system may improve usability of the drug delivery device by making pairing of the drug delivery device with a computing device more comfortable, particularly more plausible for users of the drug delivery device. Studies have shown that often users tend to use the drug injection device as a kind of input device for computing devices, particularly if the computing devices comprise touch screens. Users obviously believe that the pen-like shaped drug delivery device should work with touch interfaces, but noted that this did not work with traditional pen-like shaped drug delivery devices. With the herein disclosed system, it is however now possible for users to use a drug delivery device, which comprises attached touch input means, as input device for computing devices with touch interfaces.

In embodiments of the system, the touch interface may be a touch screen, and the pairing comprises touching a pairing button displayed on the touch screen being configured by the computer program with the touch input means. For example, the computing device may be a mobile computing device such as a smartphone, a tablet computer, a laptop computer with a touch screen, and the computer program may be a dedicated program for processing data related to a usage of the drug injection device such as an app downloadable from an app store.

In further embodiments of the system, the touching of the pairing button with the touch input means may comprise receiving and processing the touching by the computer program to switch a radio communication module of the computing device in a scan mode for detecting the electronic module within a predetermined neighbourhood area of the computing device for a certain time span and pairing with the electronic module being detected within the certain time span. Thus, a user may not take care to activate a wireless communication module of the computing device since this may be performed by the computer program once the user touches the pairing button with the touch input means. This may further increase the usability of the system for users.

In yet further embodiments of the system, the pairing may require the touching of the pairing button with the touch input means until the electronic module and the computing device are paired. For example, when the user touches the paring button with the touch input means of the drug delivery devices, the computer program may indicate to the user to continue with touching the pairing button until the pairing process is successfully terminated with both device being paired. This may help the user to better follow the pairing process since the user may note that pairing is only successfully terminated when the touching may be stopped.

In still further embodiments of the system, the drug injection device may comprise a visual indicator for signalling availability for pairing with the computing device. The visual indicator may be for example embodied by a LED (Light Emitting Diode) for visually signalling the pairing process, for example by blinking during the pairing process. In embodiments, the visual indicator may particularly comprise one or more of the following features: it may be located at the distal end of the drug injection device, particularly so that it is easily visible to users when they hold the pen in their hands. It may be controlled by the electronic module such that when the electronic module is switched into a pairing mode the visual indicator is controlled to signal the pairing mode. It may signal availability for pairing by a predetermined light sequence, for example by blinking with a relatively slow frequency during the pairing process, and by blinking with a higher frequency at the end of the successful pairing process.

A further aspect of the disclosure provides a drug injection device having a pen-like shape with a distal and a proximal end, the proximal end comprising a syringe for injecting a drug into a patient's body, the drug injection device being configured for usage with a system as described herein and comprising touch input means attached to the drug injection device and provided for making touch inputs on a touch interface of a computing device. The touch input means can e.g. be attached to the proximal end of the drug injection device. The drug injection device may be for example a Solostar™ or Allstar™ pen improved with the attached touch input means for better usability with the system as described herein.

In embodiments of the drug injection device, the touch input means may be attached to the proximal end of the drug injection device. This is particularly useful when a visual indicator for the pairing of the drug injection device with a computing device is provided at the distal end of the drug injection device so that users holding the drug injection device like a pen in their hands can easily see the visual indicator without turning the drug injection device.

A yet further aspect of the disclosure provides a cap configured to be attached to a proximal end of a drug injection device for a the syringe, wherein touch input means provided for making touch inputs on a touch interface of a computing device are attached to the cap, particularly located at the tip of the cap. The cap may be for example shaped like ballpoint pen cap so that a user may use an injection pen with the attached cap like a ballpoint for making inputs on a touch interface in a customary manner.

In embodiments of the drug injection device or the cap, the touch input means may comprise a touch pen tip. This makes it more comfortable for users to make touch inputs, for example on a smaller touch interface such as a touch display of a smartphone. Particularly, this may allow users to make writing inputs on a touch screen.

In still further embodiments of the drug injection device or the cap, the touch pen tip may be shaped like a ballpoint tip, a pencil tip or a spherical tip, and particularly the touch pen tip may comprises at least partly an elastic surface provided for making touch inputs. A ballpoint and pencil tip is particularly useful for entering handwritten data into a computing device, while a spherical tip may be more useful for activating larger buttons shown on a touch screen. The elastic surface is particularly useful to avoid damages such as scratches on the surface of a touch interface such as a touch screen.

In yet further embodiments of the drug injection device or the cap, the touch input means may be configured for usage with capacitive touch screens. In contrast to resistive touch screens, capacitive touch screens require conductive touch input means. Thus, the touch input means may at least partly be conductive in order to allow inputs on capacitive touch screens.

In further embodiments, the drug injection device may comprise an electronic module for wireless exchange of data related to a usage of the drug injection device with a device paired with the electronic module, the electronic module being attached to or integrated in the distal end of the drug injection device.

A yet further aspect of the disclosure provides a computing device being configurable for pairing with an electronic module for wireless exchange of data related to the usage of a drug injection device, the computing device comprising a touch interface and a computer program comprising instructions for execution by a processor of the computing device to configure the computing device for the pairing with the electronic module based on one or more touch inputs received via the touch interface from touch input means of a drug injection device as disclosed herein.

In embodiments of the computing device, the touch interface may be a touch screen, and the computer program may comprise instructions for execution by the processor of the computing device to display a pairing button on the touch screen, to detect a touching of the pairing button with the touch input means of the drug injection device, and to configure the computing device for the pairing with the electronic module upon detection of the touching of the pairing button with the touch input means of the drug injection device.

In further embodiments of the computing device, the computer program may comprise instructions for execution by the processor of the computing device, upon detection of the touching of the pairing button with the touch input means of the drug injection device, to switch a radio communication module in a scan mode for detecting the electronic module within a predetermined neighbourhood area around the computing device for a certain time span and pairing with the electronic module being detected within the certain time span, wherein the pairing particularly requires the touching of the pairing button until the electronic module and the computing device are paired.

### BRIEF DESCRIPTION OF THE FIGURES

FIG. 1 shows an example of a system;

FIG. 2 shows examples of drug injection devices; and

FIG. 3 shows an example of a block diagram of electronic components of the system of FIG. 1.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen.

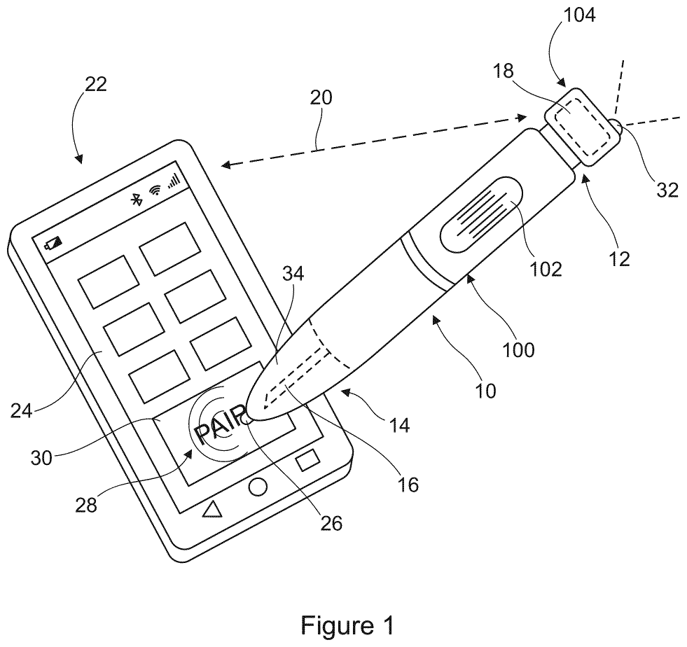

FIG. 1 shows a drug injection device 10 in the form of an injection pen and a computing device being a smartphone 22 with a capacitive touch screen 24. The injection pen 10 can be for example a reusable device such as the Allstar™ injection pen or a disposable device such as the Solostar™ injection pen. The smartphone 22 can a standard smartphone such as an iPhone™ of Apple Inc. or an Android™ powered smartphone. However, the device 22 can be any kind of mobile or stationary computing device having a touch interface such as a tablet computer, a laptop computer, a PDA (Personal Digital Assistant), a desktop computer or a dedicated computing device configured for usage with drug injection devices.

The injection pen 10 comprises a body 100 being provided for holding a drug cartridge (not shown) and housing a dose selecting and expelling mechanism (from which only a scale 102 for dose selection is shown in FIG. 1). The pen's body 100 has a distal end 12 and a proximal end 14. A syringe 16, which can be protected by a cap 34, is provided at the proximal end 14 of the body 100, and a dial grip 104, which can be used for selecting and expelling a dosage via the syringe 16 into a patient's body, is provided at the distal end 12 of the body 100.

The dial grip 104 may comprise an electronic module 18 for wireless exchange 20 of data related to a usage of the injection pen 10 with a device being paired with the electronic module 18, such as the smartphone 22. The electronic module 18 may be also configured for recording and storing dosages, and/or reading stored dosages from a memory, which were selected and expelled with the injection pen 10. The recorded and stored dosages may be comprised by the data related to the usage of the injection pen 10. The usage related data may comprise further data, for example time, date and/or amount of expelled dosages, data related to the drug such as the kind of drug, the production date, the date of expiry, and/or the date of the last use of the injection pen, a patient's identifier, a serial number of the injection pen, and any further data with any kind of relation to the usage of the injection pen 10. Thus, “data related to usage of the injection pen” as used herein must be understood as having a broad meaning in the context of this disclosure.

The electronic module 18 may be for example implement some kind of wireless connectivity such as a Bluetooth® connectivity, Near Field Communication (NFC) connectivity, and/or WiFi™ connectivity for the wireless data exchange 20. The implemented connectivity may require a pairing of the electronic module 18 with a device, with which a data exchange should be accomplished, i.e. the smartphone 22, at least one time before the data exchange can be established. This means that a wireless data exchange between the electronic module 18 and the smartphone 22 may for example not be possible in an “ad-hoc” manner without requiring any pairing. Thus, the term “pairing” as used herein can to be understood as requiring some user interaction for establishing a dedicated wireless data connection between the electronic module 18 and the smartphone 22. Particularly, pairing may comprise making a user input 28 at the touch screen 24 of the smartphone 22, particularly with a touch tip 26 arranged at the tip of the cap 34, and/or at the electronics 18. The user input 28 may comprise touching a button 28 with the touch tip 26 displayed on the touch screen 24. A user input may also be required at the injection pen 10, for example by pressing a button or turning the dial grip 104, by which the electronic module 18 is activated, i.e. powered. Particularly, the user input at the injection pen 10 may switch the electronic module 18 into a pairing mode, for example by a certain user interaction with the dial grip 104 such as for example pulling the dial grip 104 a little bit out of the body 100 of the injection pen 10 or pushing the dial grip 104 a little bit into the body 100 to switch the electronic module 18 into the pairing mode.

A visual indicator 32 such as a LED (Light Emitting Diode) may be arranged at the end of the dial grip 104. The visual indicator 32 may be controlled by the electronic module 18 to indicate the pairing mode, i.e. that the electronic module 18 is available for pairing. This indication of the pairing mode may comprise a certain colour (for example blue) and/or a certain light sequence (for example rapid blinking). The pairing availability signalled by the visual indicator 32 should be such that a user holding the injection pen 10 can easily see it.

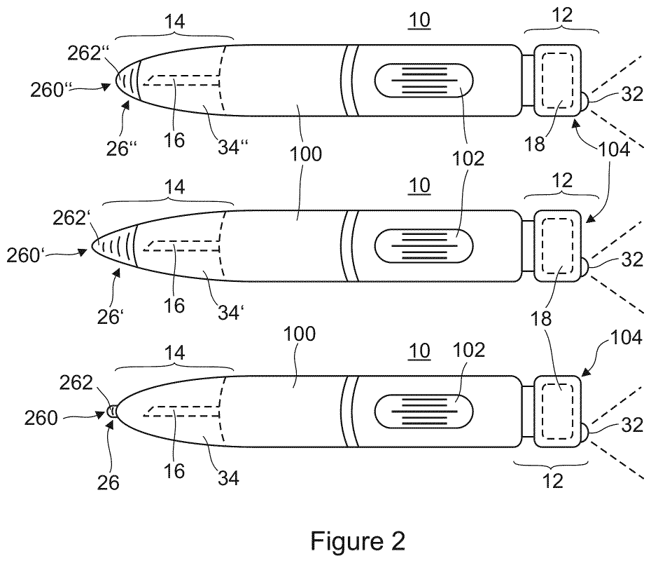

FIG. 2 shows the injection pen with different caps 34, 34′, 34″ being attached to the distal end 14 of the injection pen 10 to cover the syringe 16. The caps 34, 34′, 34″ particularly differ in the touch input means 26, 26′, 26″ provided at their tips.

The cap 34 comprises touch input means 26 with a touch pen tip shaped like a ballpoint tip 260 particularly suitable for making writing and clicking inputs on a touch screen like on the touch screen 24 of the smartphone 22 shown in FIG. 1. The shape of the ballpoint tip 260 alleviates making touch inputs on user interfaces with particularly small touch filed like small buttons, text fields etc. The ballpoint tip 260 may comprise at least partly an elastic surface 262. Particularly, the tip of the ballpoint tip 260 may form the elastic surface part 262, for example may be made from an elastic material such as rubber. The elastic surface 262 should be made particularly from a material chosen to avoid any damages of the touch screen 24, for example scratches.

The cap 34′ comprises touch input means 26′ with a touch pen tip shaped like a pencil tip 260′ particularly suitable for making clicking and also writing inputs on a touch screen like on the touch screen 24 of the smartphone 22 shown in FIG. 1. The shape of the pencil tip 260′ alleviates making touch inputs on user interfaces with particularly large and medium sized touch filed like large and medium sized buttons, text fields etc. The pencil tip 260′ may comprise at least partly an elastic surface 262′. Particularly, the tip of the pencil tip 260′ may form the elastic surface part 262′, for example may be made from an elastic material such as rubber. The elastic surface 262′ should be made particularly from a material chosen to avoid any damages of the touch screen 24, for example scratches.

The cap 34″ comprises touch input means 26 with a touch pen tip shaped like a spherical tip 260″ particularly suitable for making clicking inputs on a touch screen like on the touch screen 24 of the smartphone 22 shown in FIG. 1. The shape of the spherical tip 260″ alleviates making touch inputs on user interfaces with large touch filed like large buttons. The spherical tip 260″ may comprise at least partly an elastic surface 262″. Particularly, the tip of the spherical tip 260″ may be formed as the elastic surface 262″, for example may be made from an elastic material such as rubber. The elastic surface 262″ may be made from a material chosen to avoid any damages of the touch screen 24, for example scratches.

All three examples of the injection pen 10 shown in FIG. 2 make it more natural for a user to use the pen 10 also for making touch inputs on a touch interface such as the touch screen 24 of the smartphone 22 as shown in FIG. 1. Users tend to make touch inputs with the tip of the distal end 15 of the pen 10 on the touch screen 24, and, thus, the differently shaped touch input means 26, 26′, 26″ increase the usability of the injection pen 10.

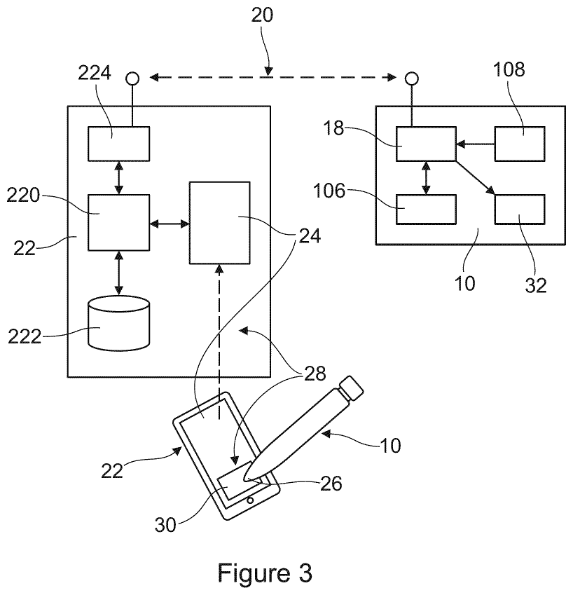

FIG. 3 shows a block diagram of the electronic components of the system of FIG. 1. The smartphone 22 comprises a processor 220 and a storage 222 accessible by the processor 220 for read/write access. The processor 220 is connected to the touch screen 24 and controls the representation on the touch screen 24 such as button, e.g. the pairing button 30, and receives touch inputs made on the touch screen 24, for example touching the pairing button 30 with the touch tip 26 of the injection pen 10. The processor 220 is further connected to a radio communication (wireless) module 224 provided for establishing radio or wireless communication connections with other devices such as the electronic module 18 of the injection pen 10. The wireless module 224 may be for example implement some kind of wireless connectivity such as a Bluetooth® connectivity, Near Field Communication (NFC) connectivity, and/or WiFi™ connectivity for the wireless data exchange 20 with another device such as the electronic module 18.

The injection pen 10 comprises the electronic module 18, and may further comprise one or more sensors 106 for acquiring data related to medications, for example selected drug dosages, which were expelled with the injection pen 10. The electronics 18 may be powered by a battery 108 such as coin cell being particularly integrated in the dial grip 104. The visual indicator 32 is controlled by the electronic module 18, particularly activated to indicate a pairing mode of the electronic module 18. The electronic module 18, the battery 108, and the visual indicator 32 may be integrated in a compact module such as on a PCB (printed circuit board) designed to be housed by the dial grip 104. The compact module may also comprise at least a part of the one or more sensors 106, for example one or more optical detectors if an optical encoding system is applied in the injection pen 10 to detect dosage selection.

The processor 220 executes a computer program stored in the storage 222. The computer program may comprise an operating system of the smartphone 22 such as for example iOS™ of Apple Inc. or Android™ of Google LLC. The computer program may comprise instructions to configure the pairing of the smartphone 22 with the electronic module 18. The pairing may be based on one or more touch inputs 28 received via the touch screen 24 from the touch tip 26 of the injection pen 10.

The pairing may be performed with functions of the operating system, for example such as a common Bluetooth® pairing or a WiFi™ direct connection. Alternatively or additionally, the pairing may be also performed by an app. For example, a user may launch an app stored in the storage 222 on the smartphone 22 and being provided for pairing with the electronic module 18 of the injection pen 10 and receiving drug related data acquired with the one or more sensors 106 via the wireless connection 20 after having been paired. The processor 220 may execute the instructions of the app to display a graphical user interface (GUI) of the app on the touch screen 24. The GUI may show one or more buttons including the pairing button 30. The processor 220 may detect touch inputs 28 made with the touch tip 26 of the injection pen 10 on the touch screen 24. Upon detection of a touch input, the processor 220 may recognize the touched button. If the processor 220 detects a touching of the pairing button 30, it may activate the wireless module 224 (if not yet active) and switch it into a scan mode, for example a Bluetooth® or WiFi™ scan mode for detecting active wireless modules such as the electronic module 18 in a predetermined neighbourhood. The neighbourhood may be predetermined by the wireless range of the wireless module 224. For example, if the wireless module 224 comprises Bluetooth® connectivity, it may be configured to detect active wireless modules in a range of about several centimetres around the smartphone 22 by broadcasting pairing requests and receiving replies to the pairing requests. The scan mode may be active for certain time span, which is limited, for example to about 30 to 60 seconds. When the wireless module 224 receives a reply its broadcast paring requests from the electronic module 18 within the time span, a pairing between the wireless module 224 and the electronic module 18 may be performed. The pairing may require that the pairing button 30 is touched until the pairing is finished with the electronic module 18 and the wireless module 224 being paired, for example that a user may hold the touch tip 26 of the injection pen 10 during the entire pairing process on the pairing button 30.

The electronic module 18 may be switched into a pairing mode by a switch (not shown). The switch may be integrated in the dial grip 104 such that it may be toggled by pushing the dial grip 104 a little bit into the pen's body 100, or pulling it a little bit out of the pen's body 100, or by turning the dial grip 104. When the electronic module 18 is switched into the pairing mode, it may control the visual indicator 32 to show a visual indication of the pairing mode, for example a blinking sequence. A user may thus recognize that the injection pen 10 or the electronic module 18 is ready for pairing and can launch the above described app on the smartphone 22 to show the GUI with the pairing button 20. Then, the user can touch the pairing button 30 with the touch tip 26 of the injection pen 10 and control the processor 220 to establish the pairing with the electronic module 18 of the injection pen 10 via the wireless module 224.

After pairing, data related to the pairing, for example unique IDs of the electronic and wireless modules 18, 224 may be stored in the storage 222 and an internal storage of the electronic module 18. The processor 220 may thereafter update the GUI to no longer show the pairing button 30, and the electronic module 18 may control the visual indicator 32 to indicate successful pairing, for example by a continuously activation. On both device 22 and 10, the pairing mode may the terminated, and the wireless connection 20 may be established for data exchange.

The processor 220 may then show further buttons on the GUI displayed on the touch screen 24, with which the user may for example initiate a data transmission. For example, the user may then touch a data request button on the GUI with the touch tip 26 of the injection pen 10, which may cause the processor 220 to control the wireless module 224 to transmit a data request command via the link 20 to the electronic module 18. The electronic module 18 can then upon receipt of the request transmit internally stored dosage related data which were acquired for example with the sensor(s) 106.

After receipt of the requested data, the user could then by touching a data display button on the GUI with the touch tip 26 of the injection pen 10 instruct the processor to update the GUI to display the received data.

All of the above described functions are only example of the intuitive handling of the entire system comprising the device 22 with the touch interface 24 and the injection pen 10 with the touch tip 26. As described above, the system enables a user to comfortably use the injection pen 10 for making touch inputs on the touch interface 24 of an external computing device 22. Particularly, the pairing of the injection pen 10 with the device 22 for data exchange may be made much more intuitive for users.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1-14. (canceled)

15. A system comprising:
a drug injection device comprising a distal end and a proximal end that comprises a syringe for injecting a drug into a patient's body;
an electronic module for wirelessly exchanging data related to a usage of the drug injection device with a device paired with the electronic module, the electronic module being attached to or integrated in the distal end of the drug injection device;
a computing device configurable for a pairing with the electronic module for wireless exchange of the data and comprising a touch interface;
a touch input means for providing inputs on the touch interface, the touch input means attached to the drug injection device; and
a computer program comprising instructions for execution by a processor of the computing device to configure the computing device for the pairing with the electronic module based on the inputs received via the touch interface.

16. The system of claim 15, wherein the touch interface comprises a touch screen, wherein the pairing comprises touching a pairing button with the touch input means, and wherein the pairing button is displayed on the touch screen and is configured by the computer program.

17. The system of claim 16, wherein the touching of the pairing button with the touch input means comprises receiving and processing the touching by the computer program to switch a radio communication module of the computing device into a scan mode for detecting the electronic module within a predetermined area of the computing device for a certain time span.

18. The system of claim 17, wherein the pairing with the electronic module is detected within the certain time span.

19. The system of claim 18, wherein the pairing comprises touching of the pairing button with the touch input means until the electronic module and the computing device are paired.

20. The system of claim 15, wherein the drug injection device comprises a visual indicator for signaling an availability for the pairing with the computing device.

21. The system of claim 20, wherein the visual indicator is located at the distal end of the drug injection device.

22. The system of claim 20, wherein the visual indicator is controlled by the electronic module such that when the electronic module is switched into a pairing mode, the visual indicator is controlled to signal the pairing mode, and/or wherein the visual indicator is configured to signal the availability for pairing by a predetermined light sequence.

23. The system of claim 15, wherein the drug injection device has a pen-like shape.

24. A drug injection device comprising a distal end and a proximal end that comprises a syringe for injecting a drug into a patient's body, wherein the drug injection device is configured for usage with a system, the system comprising:
an electronic module for wirelessly exchanging data related to a usage of the drug injection device with a device paired with the electronic module, the electronic module being attached to or integrated in the distal end of the drug injection device;
a computing device configurable for a pairing with the electronic module for wireless exchange of the data and comprising a touch interface;
a touch input means for providing inputs on the touch interface, the touch input means being attachable to the proximal end of the drug injection device; and
a computer program comprising instructions for execution by a processor of the computing device to configure the computing device for the pairing with the electronic module based on the inputs received via the touch interface.

25. The drug injection device of claim 24, further comprising a cap configured to be attached to the proximal end for covering the syringe, wherein the touch input means is attached to the cap and located at a tip of the cap.

26. The drug injection device of claim 25, wherein the touch input means comprises a touch pen tip.

27. The drug injection device of claim 26, wherein the touch pen tip is shaped like a ballpoint tip, a pencil tip, or a spherical tip, and wherein the touch pen tip comprises at least a part of an elastic surface for entering touch inputs.

28. The drug injection device of claim 24, wherein the touch input means is configured for usage with capacitive touch screens.

29. The drug injection device of claim 24, wherein the drug injection device has a pen-like shape.

30. A computing device that is configurable for a pairing with an electronic module for wirelessly exchanging data related to a usage of a drug injection device, the computing device comprising a touch interface and a computer program comprising instructions for execution by a processor of the computing device to configure the computing device for the pairing with the electronic module based on one or more touch inputs received via the touch interface from a touch input means of a drug injection device, the drug injection device comprising a distal end and a proximal end that comprises a syringe for injecting a drug into a patient's body.

31. The computing device of claim 30, wherein the touch interface comprises a touch screen, and wherein the computer program comprises instructions for execution by the processor to display a pairing button on the touch screen, to detect a touching of the pairing button with the touch input means, and to configure the computing device for the pairing with the electronic module upon detection of the touching of the pairing button with the touch input means.

32. The computing device of claim 31, wherein the computer program comprises instructions for execution by the processor upon detection of the touching of the pairing button with the touch input means, to switch a radio communication module into a scan mode for detecting the electronic module within a predetermined area around the computing device for a certain time span.

33. The computing device of claim 32, wherein the pairing with the electronic module is detected within the certain time span.

34. The computing device of claim 33, wherein the pairing comprises the touching of the pairing button until the electronic module and the computing device are paired.
